(19) United States
(12) Patent Application Publication
(10) Pub. No.: US 2001/0018577 A1
(43) Pub. Date: Aug. 30, 2001

(54) INTRAVAGINAL CATAMENIAL DEVICE

(76) Inventors: BRIAN A. FITZPATRICK, PRINCETON, NJ (US); RAYMOND J. HULL, JR, HAMPTON, NJ (US); PAUL J. SERBIAK, YARDLEY, PA (US)

Correspondence Address: AUDLEY A CIAMPORCERO JR, ONE JOHNSON & JOHNSON PLAZA, NEW BRUNSWICK, NJ, 089337003

(21) Appl. No.: 09/342,544

(22) Filed: Jun. 29, 1999

(51) Int. Cl. A61M 1/00

(52) U.S. Cl. 604/328

(57) ABSTRACT

The present invention relates to an intravaginal catamenial device having a distensible member for sealingly engaging walls of a vagina. Inflating the distensible member provides immediate expansion after insertion into the vagina, thereby minimizing the potential for any by-pass leakage of bodily fluids. The device of the present invention employs a valve mechanism capable of two-way fluid communication that enables both inflation and deflation of the device. The distensible member may be of any shape that is useful for blocking and/or collecting bodily fluids.

### FIELD OF THE INVENTION

The present invention relates to an intravaginal catamenial device for managing bodily fluids, comprising components capable of being inflated for sealingly engaging walls of the vagina during use, and then deflated prior to removing the device from the vagina.

### BACKGROUND OF THE INVENTION

Intravaginal devices for managing bodily fluids are known in the art, and generally fall into two main categories. The first category consists of one or more absorbing bodies such as tampons containing absorbent material. An example of a tampon and methods for making the same, are disclosed in Friese, U.S. Pat. No. 4,816,100. Tampons are typically manufactured from a web of absorbent fibers and compressed into a cylindrical form for inserting into a user's vaginal canal, either digitally or with the assistance of an applicator. Once inserted into the body, tampons will expand to a volume approaching its uncompressed form as fluid is absorbed. This expansion can take place in multiple directions, such as lengthwise or radially. Although tampons dominate the market of intravaginal devices, their expansion generally is controlled by absorption of bodily fluids. Prior to this expansion, the compressed tampons may allow fluid to pass between the vaginal walls and the tampon's outer surfaces.

The second category of intravaginal devices known in the art consists of collecting devices, such as those disclosed in Zoller, U.S. Pat. No. 3,845,766 and Contente et al., U.S. Pat. No. 5,295,984. Collection devices are designed to assume a normally open, concave configuration, with an open side facing a user's cervix. The collection devices may be folded, or otherwise manipulated, to facilitate insertion into the vaginal canal.

Three recently published inventions relating to collection devices, comprise inflatable components for sealingly engaging the walls of the vaginal canal. They are Zadini et al., U.S. Pat. No. 5,674,239 and PCT Pub. No. WO 99/07433, and DEKA Products Limited Partnership, PCT Pub. No. WO 99/13810. The devices disclosed in these references incorporate one-way valves that allow pressurized air to enter the device during inflation, but do not allow the air to escape for deflating the devices. Due to this directional limitation, additional design features or techniques must be included to help in removing the devices after their useful life.

Zadini et al., U.S. Pat. No. 5,674,239, discloses an intravaginal balloon for blood leakage prevention, with two alternative methods for removal after use. The first method disclosed consists of pulling on an attached removal means, such as a string, sufficient to decrease a transverse diameter of the balloon. The second method employs microporous material for constructing the balloon. This allows air to escape at a substantially predetermined time following inflation of the distensible member.

Zadini et al., PCT Pub. No. WO 99/07433, discloses an intravaginal balloon for blood leakage prevention. A method of deflating the intravaginal balloon consists of creating a slit in a chamber wall of the balloon with the removal string, thereby providing an escape route for carbon dioxide or suitable inflating gas.

DEKA Products Limited Partnership discloses a catamenial collector having a receptacle with a flexible hollow rim capable of inflation. The device employs tear marks along portions of a handle extending from the receptacle, whereby portions of the device can be torn away, allowing for deflation prior to removal.

What is still needed, is an intravaginal catamenial device comprising an distensible member and a single valve, the valve capable of repeated two-way fluid communication providing a means for both inflation and deflation of the device.

### SUMMARY OF THE INVENTION

The present invention relates to an intravaginal catamenial device having a distensible member for sealingly engaging walls of a vagina. Inflating the distensible member provides immediate expansion after insertion of the device into the vagina, thereby minimizing the potential for any by-pass leakage of bodily fluids. The device of the present invention employs a two-way valve mechanism that is capable of maintaining an inflated pressure while being easily manipulated to reduce or eliminate the pressure, for easy and comfortable device removal.

Accordingly, there has now been provided a catamenial device for insertion into a vaginal canal, having a wall, the device comprising: a distensible member to sealingly engage the wall of the vaginal canal upon inflation, having a cervix-facing side and an opposing side; a conduit for conducting fluid in fluid communication with the distensible member extending from the opposing side; and a valve capable of repeated two-way fluid communication coupled to the conduit; wherein at least portions of the conduit proximal the valve are flexible and resilient.

The distensible member may be repeatedly inflated and deflated, through manipulation of the valve, to provide single or multiple uses, as well as to provide repositioning if physical or psychological discomfort occurs after the device is inserted. The distensible member may be of any shape that is useful for blocking and/or collecting bodily fluids. Collection of bodily fluids can take place by incorporating at least one reservoir proximal the cervix-facing side of the device.

The distensible member may be used in conjunction with absorbent bodies, comprise absorbent material on one or more of its outwardly disposed surfaces, or be used alone. The addition of absorbent material in any form, provides for improved retention of captured fluids, and thereby minimizes the potential of fluid spillage during and after removal of the used device.

### BRIEF DESCRIPTION OF THE DRAWINGS

FIG. 1 is a perspective view of an intravaginal catamenial device provided by the present invention.

FIG. 2 is a cross section of the device shown in FIG. 1 along line 2-2.

FIG. 3 is a perspective view of duckbill valve capable of two-way fluid communication useful in the present invention.

FIG. 4 is an end view of the valve shown in FIG. 3, being manipulated from a closed position to an open position.

FIG. 5 depicts a cross sectional view of a device provided by the present invention that has been inserted into a vaginal canal of a user.

### DETAILED DESCRIPTION OF THE PREFERRED EMBODIMENTS

FIG. 1 depicts an intravaginal catamenial device 10 provided by the present invention. The device 10 comprises a distensible member 20 having a cervix-facing side 21 and an opposing side 22, and a fluid conducting conduit 30 extending form the opposing side 22 that is in fluid communication with the distensible member 20. The conduit 30 and the distensible member 20 have a combined length L. Length L is preferably at least the length of a typical user's vagina, so that a user may utilize the conduit 30 for inserting, positioning, and removing the device 10. Like designations are used to designate substantially identical elements in the Drawings throughout the remainder of the specification.

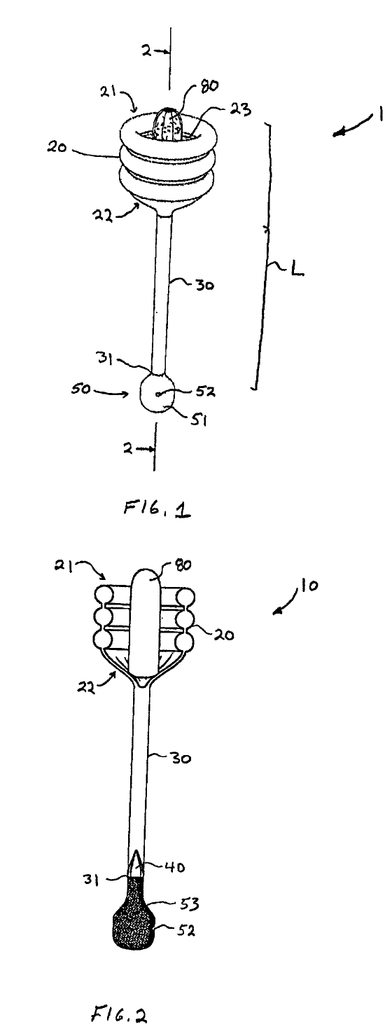

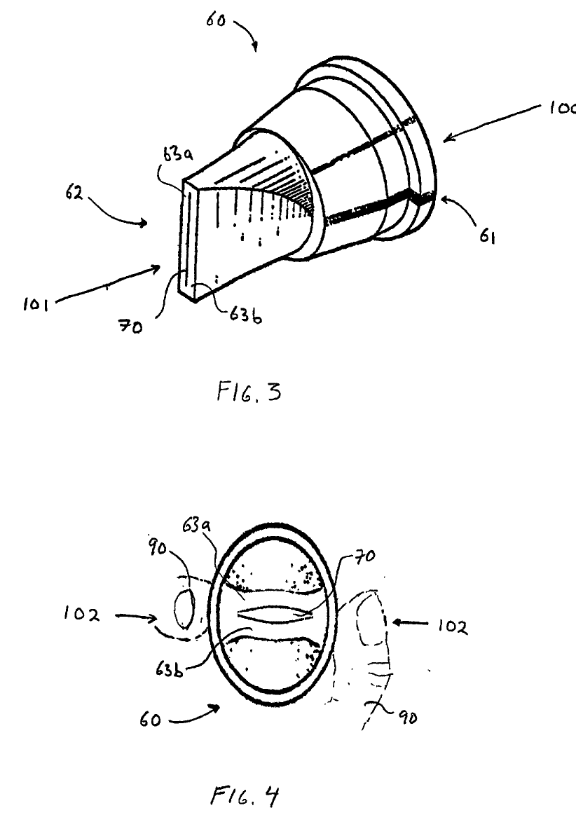

FIG. 2 is a cross section of the device in FIG. 1 taken along line 2-2, depicting a valve 40 that is capable of two-way fluid communication coupled to the conduit 30. The valve 40 is coupled to the conduit 30, meaning that it may reside substantially within the conduit, or have portions of the conduit connected to at least one end of the valve. The coupling is sealingly engaged so as to prevent any leakage of an inflation medium.

The device 10 may be inflated and deflated through the valve 40, and therefore it does not require additional elements for one function or the other. As used herein, the term “inflate” and variants thereof, include the use of both liquids and gases. The valve of the present invention also provides for repeated inflation within a short time period. As stated in the background section of the instant specification, the collection devices comprising inflatable members currently known in the art require destructive or time delayed techniques for deflation (i.e., via tear away portions or construction with microporous materials).

There are several benefits associated with the capability of repeated inflation within a short time period. First, a user may wish to reposition the device after it has been inserted and inflated, due to a sense of discomfort in the placement of the device. Second, the user may wish to deflate the device to a reduced volume sufficient to drain any bodily fluids into a disposal means, such as a toilet, and then to re-inflate it for additional use. Third, the user may find that the initial inflation is too great and may desire to slightly reduce the pressure within the distensible member without completely deflating it.

Valves capable of two-way fluid communication include both two-way valves and releasable one-way valves. Ball valves, stop cock valves, and gate valves are examples of two-way valves, wherein the valve may be manipulated to either an open or a closed position, but is not biased to either one of the positions. Check valves, reed valves, umbrella valves, and duckbill valves are examples of one-way valves. One-way valves are normally in a closed position, allowing pressurized fluid to flow in one direction, while resisting flow in the opposite direction. When one-way valves are used in the present invention, it is critical that they are accessible and manipulatable by the user, so that the valves may be releasable, thereby providing two-way fluid communication. For example, having the capability of manually compressing the spring in a spring-loaded check valve, or compressing peripheral portions of a duckbill valve sufficient to urge normally closed sealing lips apart.

In addition to the properties of the valve itself, manipulatable may also require environmental properties. What is meant by “environmental properties” is the condition or circumstances corresponding to the materials or elements of the device surrounding the valve. This includes, but is not limited to, physical properties of the valve housing and valve placement within the housing, such as depth. For example, if a duckbill valve resides within a fluid conducting conduit, then the conduit must be sufficiently flexible and resilient, in at least portions proximal the duckbill valve, so as to permit transfer of compressive forces applied to the outer surface of the conduit, to open the normally closed sealing lips. Upon release of the compressive forces, any environmental changes, such as distortion of the conduit in areas proximal the valve dissipates, due to its resiliency. This dissipation allows for repeated manipulation of valves used in the present invention.

Employment of valves that are not accessible and manipulatable, render the present invention inoperable according to its intended use, that is, for providing a means through which the distensible member may be inflated and deflated. As an example, following from above, if a duckbill valve were residing within a rigid conduit, then compressive forces applied to outer surfaces of the rigid conduit would not be sufficiently transferable to open the normally closed lips to release inflation medium occupying the distensible member, unless there were some feature extending from the valve that allowed manipulation of the valve, or the valve could be manipulated by inserting a mechanical means, such as a rod, through the conduit and into the valve to open the normally closed lips.

Inflation medium for inflating the distensible member may be any suitable fluid, including liquids and gases. A representative, non-limiting list of suitable fluids, includes saline solution, water, air, carbon dioxide, and inert gas.

An inflation medium source may be permanently or releasably attached to the device. “Source”, as used herein, includes an inflation medium generating mechanism such as a pump, as well as contained volumes of fluid. Referring again to FIG. 1, the device 10 for example depicts optional inflating mechanism 50 connected to a terminal point 31 of the conduit 30. The inflating mechanism 50 is preferably a manually operated pump, comprising a pump chamber 51 and an aperture 52 that is open to the environment.

To operate the inflating mechanism 50, a user covers the aperture 52 and then depresses the pump chamber 51, forcing air occupying the pump chamber 51 through the valve (shown in FIG. 2), and into the conduit 30. To replenish the pump chamber 51 with a new volume of air, the aperture 52 is uncovered, and air is drawn from the environment and into the pump chamber 51 due to a pressure differential. In an effort to improve the efficiency of replenishing the pump chamber 51, resilient material (shown as 53 in FIG. 2) such as open celled foam, may be included within the bladder. An example of such a resilient material is disclosed in Ahr et al., U.S. Pat. No. 5,582,604. To eliminate requirement of covering and uncovering an aperture, a one-way valve may optionally be employed in association with the pump chamber 51. The steps of depressing the pump chamber 51 and replenishing it are repeated to inflate the distensible member 20, to sealingly engage a user's vaginal walls. Equivalent manually operated pumps may alternatively be incorporated to provide pressurized air to the device.

The distensible member may be of any shape to block and/or collect discharged bodily fluids. Distensible member 20 in FIG. 1, employs a single reservoir 23 directed towards the cervix-facing side 21. Additional reservoirs may be used to improve the fluid management capabilities of the device, or alternatively be used for delivering various additives. A representative, non-limiting list of potential additives includes medicaments, moisturizers, vitamins and minerals, and odor controlling agents.

Absorbent material may optionally be associated with the distensible member for improving the fluid management capabilities of the device, such as in an overlying position on at least a portion of the distensible member's outer surface. FIG. 1 depicts a singular absorbent body 80, such as a commercially available tampon, nested within the reservoir 23. Alternative forms of absorbent bodies and materials may also be used, such as for example superabsorbent particles or fibers adhered to at least a portion of the outwardly disposed surface of the distensible member. The use of construction adhesives well known in the industry, such as styrenic block copolymers, is one suitable method for adhering absorbent material to the distensible member. Another suitable form of absorbent material is a nonwoven web comprising a blend of cellulosic fibers such as rayon and thermoplastic fibers in an amount sufficient to provide heat sealability to the outwardly disposed surface of the distensible member. A representative, non-limiting list of useful absorbent material includes natural cellulosics, regenerated cellulosics, polyacrylates, grafted starch, grafted cellulose, and polyvinyl alcohol. Cotton, wood pulp, and peat moss are examples of natural cellulosics, while rayon is an example of a regenerated cellulosic.

Preferably the valve 40 used in the device of the present invention, is a releasable one-way valve, and more preferably a one-way duckbill valve. An example of a duckbill valve and method of manufacture is disclosed in Hoffman, U.S. Pat. No. 4,524,805. FIG. 3 depicts a duckbill valve 60 having a first open end 61 progressively narrowing to a second end 62 having a pair of opposed sealing lips 63a and 63b defining a normally closed slit 70

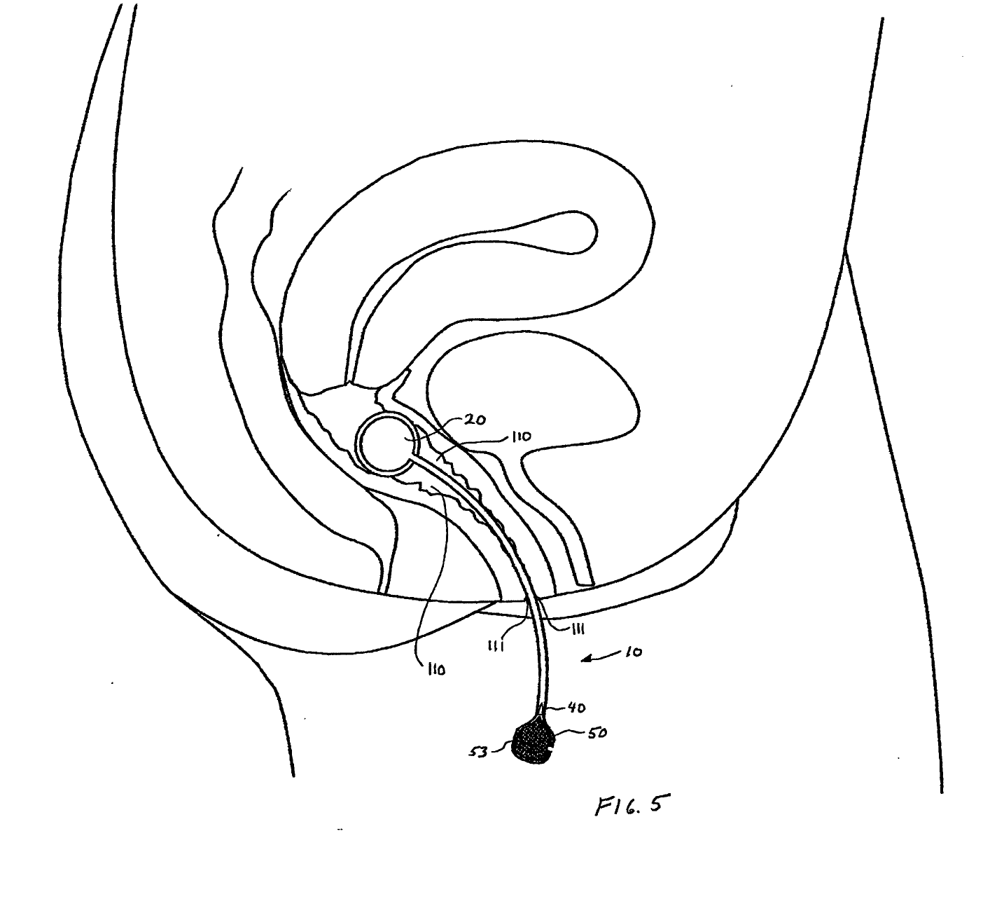

The duckbill valve 60 provides two-way fluid communication. In a first fluid direction 100, air is introduced into the open end 61 having a pressure that is greater than that outside the lips 63a and 63b, thereby urging the lips apart and opening slit 70. To impart fluid flow in a second fluid direction 101, compressive forces are applied to peripheral potions of the duckbill valve 60, substantially perpendicular to the slit 70, sufficient to urge the lips 63a and 63b apart. FIG. 4 shows a duckbill valve 60 in a partially open position through the application of a compressive force 102 by a user's manual digits 90. It should be readily understood by a person having ordinary skill in that art, that although the compressive forces 102 are depicted in FIG. 4 as being directly applied to the duckbill valve 60, transferable compressive forces may act on the valve through application to outer surfaces of various valve housing means, such as a fluid conducting conduit shown in FIGS. 1-2 and 5.

Referring now to FIG. 5, to use one of the embodiments provided by the present invention, a user inserts the device 10 into her vaginal canal 110, and then manually operates the inflating mechanism 50 to inflate the distensible member 20 (shown having a spherical shape). When the user is ready to remove the device 10, the user manipulates the valve 40 from a closed position to an open position, thereby releasing the inflation medium and deflating the distensible member 20. The user may choose to completely, or alternatively, partially deflate the distensible member 20 to facilitate an easy and comfortable removal from the vaginal canal 110.

The valve 40 is preferably positioned external to an opening 111 of the vaginal canal. External includes positions adjacent the opening 111, such as within the user's labia minora and/or majora. Valve 40 may also be positioned within the user's vaginal canal 110.

An external placement of the valve provides for preferred inflation medium exhaustion to locations outside of the body. Some inflation mediums may be useful for inflating the distensible member, however they may not be suitable for release into the body. For example, there are a number of publications suggesting that high oxygen and carbon dioxide levels within a user's vaginal canal may increase the chances of contracting toxic shock syndrome.

The distensible member may be manufactured from materials known to one having ordinary skill in the art. Suitable materials for the distensible member include, but are not limited to, latex, silicone, polyurethanes, polyvinyl chlorides, and other elastomeric materials. Inelastic materials, such as polyester terephthalate may also be used. One suitable method for making the distensible member consists of the following steps: dipping a mandrel into uncured material, curing the material on the mandrel, and then removing the formed distensible member from the mandrel.

The fluid conducting conduit may be made from any flexible material such as polyethylene, and initially formed as a substantially tubular element, through extrusion or injection molding techniques, or formed through secondary operations from a sheet of material. The distensible member, the fluid conducting conduit and the valve are preferably manufactured separately and then assembled together into a final form as shown in the figures. A representative, non-limiting list of assembly techniques and materials, includes adhesives, heat seal, ultrasonic welding, solvent welding, and mechanical fastening.

The disclosures of all U.S. patents and patent applications, as well as any corresponding published foreign patent applications, mentioned throughout this patent application are hereby incorporated by reference herein.

While particular embodiments of the present invention have been illustrated and described, other changes and modifications can be made without departing from the spirit and scope of the invention. It is therefore intended to cover in the appended claims all such changes and modifications that are within the scope of this invention.

What is claimed is:

1. A catamenial device for insertion into a vaginal canal, having a wall, the device comprising:
a) a distensible member to sealingly engage the wall of the vaginal canal upon inflation, having a cervix-facing side and an opposing side;
b) a conduit for conducting fluid in fluid communication with the distensible member extending from the opposing side; and
c) a valve capable of repeated two-way fluid communication coupled to the conduit.

2. The device of claim 1 wherein at least portions of the conduit proximal the valve are flexible and resilient.

3. The device of claim 1 further comprising an inflation medium source positioned at a terminal point of the conduit distal the distensible member.

4. The device of claim 3 wherein the inflation medium source is a manually actuated pump having a bladder, a resilient material within the bladder, and at least one aperture through a wall of the bladder.

5. The device of claim 1 wherein inflation medium for inflating the distensible member is selected from the group consisting of air, carbon dioxide, saline solution, water, and inert gas.

6. The device of claim 1 wherein the distensible member is capable of being deflated through manipulation of the valve from a closed position to an open position, thereby allowing inflation medium occupying the distensible member to escape through the valve.

7. The device of claim 6 wherein deflation of the device exhausts the inflation medium to a location external to the vaginal canal.

8. The device of claim 1 wherein the valve is positioned external to an opening of the vaginal canal.

9. The device of claim 1 wherein the valve is a two-way valve.

10. The device of claim 1 wherein the valve is a releasable one-way valve.

11. The device of claim 10 wherein the releasable one-way valve is a duckbill valve comprising a first open end narrowing to a second end having a pair of opposed sealing lips defining a normally closed slit.

12. The device of claim 11 wherein the distensible member is capable of being deflated by applying compressive forces to peripheral portions of the duckbill valve, substantially perpendicular to the slit, sufficient to separate the sealing lips, thereby allowing inflation medium occupying the distensible member to escape through the valve.

13. The device of claim 12 wherein the distensible member is capable of being re-inflated by removing the force on the valve and re-introducing inflation medium into the distensible member.

14. The device of claim 1 further comprising absorbent material associated with outwardly disposed surfaces of the distensible member.

15. The device of clam 14 wherein the absorbent material is selected from the group consisting of natural cellulosics, regenerated cellulosics, polyacrylates, grafted starch, grafted cellulose, and polyvinyl alcohol.

16. A catamenial device for insertion into a vaginal canal, having a wall, the device comprising:
a) a distensible member to sealingly engage the wall of the vaginal canal upon inflation, having a cervix-facing side and an opposing side;
b) a conduit for conducting fluid in fluid communication with the distensible member extending from the opposing side;
c) a valve capable of repeated two-way fluid communication coupled to the conduit; and
d) at least one reservoir proximal the cervix-facing side for collecting bodily fluids.

17. The device of claim 16 wherein at least portions of the conduit proximal the valve are flexible and resilient.

18. The device of claim 16 further comprising an inflation medium source positioned at a terminal point of the conduit distal the distensible member.

19. The device of claim 18 wherein the inflation medium source is a manually actuated pump having a bladder, a resilient material within the bladder, and an at least one aperture through a wall of the bladder.

20. The device of claim 16 wherein inflation medium for inflating the distensible member is selected from the group consisting of air, carbon dioxide, saline solution, water, and inert gas.

21. The device of claim 16 wherein the distensible member is capable of being deflated through manipulation of the valve from a closed position to an open position, thereby allowing inflation medium occupying the distensible member to escape through the valve.

22. The device of claim 21 wherein deflation of the device exhausts the inflation medium to a location external to the vaginal canal.

23. The device of claim 16 wherein the valve is positioned external to an opening of the vaginal canal.

24. The device of claim 16 wherein the valve is a two-way valve.

25. The device of claim 16 wherein the valve is a releasable one-way valve.

26. The device of claim 25 wherein the releasable one-way valve is a duckbill valve comprising a first open end narrowing to a second end having a pair of opposed sealing lips defining a normally closed slit.

27. The device of claim 26 wherein the distensible member is capable of being deflated by applying compressive forces to peripheral portions of the duckbill valve, substantially perpendicular to the slit, sufficient to separate the sealing lips, thereby allowing inflation medium occupying the distensible member to escape through the valve.

28. The device of claim 27 wherein the distensible member is capable of being re-inflated by removing the force on the valve and re-introducing inflation medium into the distensible member.

29. The device of claim 16 further comprising absorbent material within the at least one reservoir.

30. The device of claim 29 wherein the absorbent material is selected from the group consisting of natural cellulosics, regenerated cellulosics, polyacrylates, grafted starch, grafted cellulose, and polyvinyl alcohol.

31. A method of managing bodily fluids during a period of menstruation, comprising the steps of:
a) providing a catamenial device for insertion into a vaginal canal, having a wall, the device comprising
i) a distensible member to sealingly engage the wall of the vaginal canal upon inflation, having a cervix-facing side and an opposing side;
ii) a conduit for conducting fluid in fluid communication with the distensible member extending from the opposing side; and
iii) a valve capable of repeated two-way fluid communication coupled to the conduit;
b) inserting the distensible member into the vaginal canal;
c) inflating the distensible member sufficient to sealingly engage the wall of the vaginal canal;
d) maintaining the catamenial device in the vaginal canal for preventing passage of the bodily fluids external the vagina for a duration of at least about 2 hours;
e) manipulating the valve to an open position for deflating the distensible member to a reduced volume sufficient to drain the bodily fluids; and
f) repeating steps (c) through (e) as needed during the menstrual period.

32. The method of claim 31 wherein the catamenial device further comprises at least one reservoir proximal the cervix-facing side for collecting the bodily fluids.

33. The method of claim 31 wherein the step of deflating the distensible member exhausts inflation medium occupying the distensible member external to the vaginal canal.

34. The method of claim 31 wherein the valve is a releasable duckbill valve having a pair of opposed sealing lips defining a normally closed slit, and step (e) comprises applying compressive forces to the conduit proximal the valve sufficient to separate the sealing lips.

35. A method of managing bodily fluids during a period of menstruation, comprising the steps of:
a) providing a catamenial device for insertion into a vaginal canal, having a wall, the device comprising
i) a distensible member to sealingly engage the wall of the vaginal canal upon inflation, having a cervix-facing side and an opposing side;
ii) a conduit for conducting fluid in fluid communication with the distensible member extending from the opposing side; and
iii) a valve capable of repeated two-way fluid communication coupled to the conduit;
b) inserting the distensible member into the vaginal canal;
c) inflating the distensible member sufficient to sealingly engage the wall of the vaginal canal;
d) maintaining the catamenial device in the vaginal canal for preventing passage of the bodily fluids external the vagina for a duration of at least 2 hours;
e) manipulating the valve to an open position for deflating the distensible member; and
f) removing the catamenial device from the vaginal canal.

36. The method of claim 35 wherein the catamenial device further comprises at least one reservoir proximal the cervix-facing side for collecting the bodily fluids.

37. The method of claim 35 wherein the step of deflating the distensible member exhausts inflation medium occupying the distensible member external to the vaginal canal.

38. The method of claim 35 wherein the valve is a releasable duckbill valve having a pair of opposed sealing lips defining a normally closed slit, and step (e) comprises applying compressive forces to the conduit proximal the valve sufficient to separate the sealing lips.
